(19) United States
(12) Patent Application Publication
(10) Pub. No.: US 2008/0200539 A1
(43) Pub. Date: Aug. 21, 2008

(54) Topical antifungal treatment

(76) Inventor: Alexander A. Bommarito, Freeland, MI (US)

Correspondence Address: Alexander D. Bommarito, 12555 W. Freeland Road, Freeland, MI, 48623, US

(21) Appl. No.: 12/148,302

(22) Filed: Apr. 18, 2008

Related U.S. Application Data
(63) Continuation-in-part of application No. 10/289,552, filed on Nov. 7, 2002, now Pat. No. 7,374,772.

(51) Int. Cl. A61K 31/343; A61K 31/225; A61P 31/10; A61K 31/27

(52) U.S. Cl. 514/462; 514/547; 514/479

(57) ABSTRACT

The present invention is a topical skin preparation for treatment of fungal infections of the skin and nails. The preparation comprises triacetin in combination with one or more antifungal agents. In a preferred form, the preparation comprises a combination of the antifungal agents tolnaftate and grisiofulvin are used in combination with triacetin. The concentrations of these constituents are 40 to 50% concentration triacetin, 30 to 50% acetone, 0.5-3.0% concentration tolnaftate and 0.5-4.0% concentration grisiofulvin, in one preferred embodiment. Other compounds, such as ethyl alcohol, acetone, amino acids such as n-acetylcysteine, and herbal additives may also be added to the preparation. Further, other antifungal agents such as nystatin, clortimazole, econazole, ketoconazole, miconazole, solconazole, oxiconizole, naftifine, terbinafine, and butenafine, for example, may be substituted for the antifungal agents tolnaftate and grisiofulvin. The preparation of the present invention is effective in treating immune compromised patients and those with diabetes, as well as relatively healthy persons.

### CROSS-REFERENCE TO RELATED APPLICATION

This application is a continuation-in-part application of application Ser. No. 10/289,552, filed Nov. 7, 2002.

### FIELD OF THE INVENTION

The present invention is generally a topical skin treatment. More specifically, the present invention is a topical skin treatment effective at treating skin and nail infections such as onychomycosis, Tinea pedis, Tinea cruris, Tinea corporis, Tinea versicolor and Candidiasis, among others.

### BACKGROUND OF THE INVENTION

Fungal skin infections, often called dermatomycoses, are among the most common skin infections. Characteristically, these infections exhibit single or multiple lesions that may produce a mild scaling, or deep, inflamed, nodular lesions. Many of these infections are superficial, affecting the hair, nails, and/or skin, and are generally caused by three fungi: Trichophyton, Microsporum, and Epidermophyton.

Since clinical differentiation of the similar dermaphytes is difficult, these infections are discussed or grouped and treated according to the sites involved. Diagnosis of difficult, non-responding pathogenic organisms can be identified by scraping of the developed lesions, and examining by either direct microscopic examination of a potassium hydroxide preparation or by fungal culture.

Some of the fungal skin infections are classed as follows. Tinea Pedis is a very common fungal infection, which is commonly known as athletes foot or ringworm of the feet. Tinea pedis can be difficult to cure in some affected persons.

Tinea curis is commonly known as jock itch, and is caused by Epidermophyton floccosum, Trichophyton rubrum, or Trichophyton mentagrophytes. They occur on the medial and upper portions of the thighs, and in the pubic area. Tinea curis is more commonly seen in males. Tinea curis lesions have specific margins with small vessels commonly found. Acute lesions are bright red, and chronic, recurring cases tend to be hyper pigmented.

Tinea corporis is known as ringworm of the skin and is more prevalent in climates with higher humidity. Tinea corporis usually involves infections by Trichophyton or Microsporum species. Lesions causes by Tinea corporis form on smooth and bear skin areas. These lesions begin as small circular red areas and can become scaling, raised, and pruritic areas.

Tinea versicolor is a fungal infection that generally creates cosmetic concerns. Lesions generally occur on seborrheic areas in a confetti-like configuration. This common superficial fungal infection of the stratum corneum is caused by Ptiyrosporum orbiculare.

Candidiasis or moniliasis is caused primarily by Candida albicans, and usually occurs in the groin, axilla, interdigital spaces, and under the breasts.

Onychomycosis, or fungal infections of the nail bed or plate, contribute to 40% of all nail disorders. It has been estimated that total United States Medicare costs for treating onychomycosis are in excess of 43 million dollars. Mycotic nail infections are most commonly caused by dermatophytes (Trichophyton, Microsporum, and Epidermophyton species), yeasts (Candida species), and nondermatophyte molds (Scytalidium, Fusarium, Acremonium, Aspergillus and Scopulariopsis, species). Four major types of mycotic nail infections have been identified: distal subungual onychomycosis, which is the most common type, affecting the plantar surfaces of the hands and feet; white superficial onychomycosis, which affects the toenails; proximal subungual onychomycosis, which is often associated with immunosuppression; and, candidal onychomycosis. These infections vary with respect to the pattern of fungal invasion and causative pathogen. Clinical symptoms of onychomycosis include onycholysis, or separation of the nail from its bed, hyperkeratosis such as calluses or corns, brittleness, color change, and paronychial inflammation, or inflammation due to infection of the skin fold at the nail margin.

Mycotic nail infections do not always resolve spontaneously and may have serious consequences, including limitation of mobility and dexterity, decrease in peripheral circulation in the affected area, and self-consciousness. Onychomycosis can also worsen pre-existing foot problems, such as problems caused in diabetic patients.

Numerous different topical preparations, both prescription and non-prescription, have been used to treat these fungal infections. A significant number of these preparations are sold over-the-counter in drug stores, grocery stores and other retail outlets. On Feb. 26, 1999 the United States Food & Drug Administration identified certain topical antifungal agents that could be generally recommended as safe and effective for non-prescription use, and other agents that could not be recommended. The agents identified as topical non-prescription antifungal agents that are recognized as safe and effective included clioquinal, providone iodine, clortrimazole, tolnaftate, haloprogin, undecylenates, and miconazole nitrate. Those identified as not generally recognized as safe and effective included, alcloza, alum potassium, aluminum sulfate, amyltricresols, basic fuchsin, benzethomium chloride, benzoic acid, benzoxiquine, boric acid, camphor, candicidin, chlorothymol, coal tar, dichlorophen, menthol, methylparaben, oxyquinoline, oxyquinoline sulfate, phenol, phenolate sodium, phenyl salicylate, propionic acid, propylparaben, resorcinol, salicylic acid, sodium borate, sodium caprylate, sodium propionate, sulfur, tannic acid, thymol, tolindate, triacetin, zinc caprylate, zinc propionate.

Even the effective agents are not effective in all conditions. In treating patients with immune suppressed disorders such as diabetes, chemotherapy patients, HIV infected patients, and those with chronic infections, fungal infections go untreated or unsuccessfully treated.

In attempting to achieve optimum blood sugar levels in controlling diabetes, infections must be controlled. Infections raise blood sugar levels. Patients with diabetes are generally on a number of medications, including antidiabetic agents, antihypertensives, and lipid lowering drugs. Oral antifungal agents compete with these other medications at the liver for removal from the body. This activity leads to elevated liver enzyme levels. Once liver enzyme levels are elevated, patients have to be taken off of their antifungal and other medications. The same situation holds true with other immunosuppressed patients, including chemotherapy patients and HIV affected patients. Further, drug interactions have to be monitored during treatment with oral antifungal agents.

Accordingly, there is a need to provide an antifungal preparation capable of treating various fungal infections that will effectively work in medically compromised, as well as relatively healthy persons. A topical preparation is the best for this situation, as liver complications are avoided.

### SUMMARY OF THE INVENTION

The present invention is a topical antifungal preparation for treatment of fungal infections of the skin and nails. At a minimum, the preparation of the present invention comprises triacetin and an antifungal agent such as tolnaftate, for example. In a preferred form, the preparation comprises triacetin, a fatty acid source such as a fish oil, and an antifungal agent. In a preferred embodiment, the preparation is comprised of 96 to 99% triacetin, 0.5 to 1% cod liver oil and 1 to 3% tolnaftate. In another preferred embodiment, the preparation is comprised of 40 to 50% triacetin, 30 to 50% acetone and 0.5 to 4.0% grisiofulvin. It is to be understood that the antifungal agents tolnaftate and grisiofulvin are only preferred examples of the antifungal agents that can be utilized with this invention. Tolnaftate, triacetin and grisiofulvin are used both because of their antifungal characteristics, as well as the fact that they are safe if improperly or mistakenly ingested by patients. Other agents, such as amino acids, alcohol and garlic oil may also selectively be incorporated into the preparation to aid in its effectiveness. In use, preparation of the present invention is applied topically to the affected area(s) until the infection has been resolved.

### DETAILED DESCRIPTION OF THE PREFERRED EMBODIMENTS

Reference will now be made in detail to the present preferred embodiments of the present invention. The present invention is a novel topical antifungal preparation comprised of triacetin in combination with an antifungal agent. In a preferred composition, the preparation is comprised of triacetin, an antifungal agent, and a fish oil or like fatty acid source. In a preferred embodiment, the preparation is comprised of triacetin, cod liver oil and tolnaftate. In one preferred embodiment, this preparation is comprised of 96 to 99% triacetin, 0.5 to 1% cod liver oil, and 0.5 to 3% tolnaftate. In another preferred embodiment, the preparation is comprised of 40 to 50% triacetin, 30 to 50% acetone and 0.5 to 4.0% grisiofulvin. It is to be understood that the antifungal agents tolnaftate and grisiofulvin are only preferred examples of the antifungal agents that can be utilized with this invention. Tolnaftate, triacetin and grisiofulvin are used both because of their antifungal characteristics, as well as the fact that they are safe if improperly or mistakenly ingested by patients.

Other fish oils, such as mandarin oil, shark liver extract, salmon oil, and purified Omega-3 fatty acid fish oils such as OMEGA PURE produced by Omega Protein, Inc. of Reedsville, Va., may be used instead of cod liver oil. However, cod liver oil is beneficial in aiding to heal the skin due to its high levels of vitamins A and D, as well as being relatively inexpensive. Further, other oils, triglicerides, or free fatty acids having similar characteristics to fish oils being high in Omega-3 fatty acids may also be used. Fatty acids aid in antifungal activity. One problem in patients with fungal infections is decreased fatty acid production by the sebaceous glands, due to various causes. By introducing fatty acids to the infected area(s), a more healthy skin environment is restored. Synthetic vitamin A may also be used and works like fish oil from my experiments and trials, due to a deficiency of vitamin A from fungal antimetabolite activity. The yellow color seen in nail infections, or onychomycosis, is caused by the inability of vitamin A precursors being precluded from vitamin A formation and function due to the infection.

Tolnaftate is used topically for the treatment of certain dermatophytoses, such as Tinea cruris, Tinea corporis, Tinea manuum, and Tinea pedis, which are caused by Trychophyton rubrum, Trychophyton tonsurans, Microsporum canis, Microsporum audouini, or Epidermophyton floccosum. The exact mode of action of tolnaftate is not known, but the drug has been demonstrated to distort the hyphae and stunt mycelial growth in susceptible fungi. In vitro, tolnaftate is fungistatic or fungicidal to Microsporum gypseum, Microsporum canis, Microsporum audouini, Microsporum japonicum, Trichophyton rubrum, Trichophyton mentagrophytes, Trichophyton schoenleinii, Trichophyton tonsurans, and Epidermophytom flocossum. Activity of tolnaftate against Aspergillis niger varies from one strain to another.

Triacetin is an odorless, stainless and non-allergenic liquid. Triacetin is an antifungal drug proposed for use in the treatment of superficial fungal infections of the skin, particularly for Trichophyta, Epidermophyta, and Microspora. The hydrolysis of the ester by myco enzymes releases acetic acid, and any antifungal or antimicotic activity is attributable to the drop in pH. Previous clinical studies by others suggest that the drug is only mildly effective as an antifungal agent when used alone.

In the present preparation, a chemically pure 98 to 99% glyeryl triacetate, formula weight 218.2, increases the activity of the antifungal agent, such as tolnaftate. The increase in activity is accomplished by providing optimal skin exposure by the antifungal agent. Triacetin is used in the cosmetic and food industries for its ability to act as a carrier or vehicle. In this preparation, triacetin acts as a carrier for the antifungal agent, while also increasing the effectiveness of the antifungal agent.

Acetone is selectively used when grisiofulvin is the selected antifungal agent. Acetone is a well known solvent with a variety of uses and advantages. With its low boiling point, acetone is used with the present invention to improve solubility and stability of the preparation, as well as aid in reducing the moisture content within the infected tissue.

Triacetin also acts to decrease the pH of the application area It is known that fungus like moist areas and thrive in environments with a normal acidity, pH 6.5 to 7.2. Yeasts die in low pH environments, i.e. pH levels in the range of 4.5 or less, and infectious fungi are adversely affected in low pH environments. Triacetin has a pH of 4.0 when applied to the skin, and effectively lowers the pH of the application area. As triacetin breaks down after application, it produces acetic acid, a weak antifungal agent. Triacetin also acts to lower the moisture content in the application area, which aids in creating an adverse environment for fungal growth. In high concentrations, such as in this preparation, triacetin reduces the secretion of moisture during perspiration, due to an astringent effect, or antihyperhydrosis effect. Triacetin also acts to reduce moisture by bonding with the moisture available in the affected area, reducing the opportunity or availability of the moisture to be utilized by the fungal cells. After application, triacetin also coats the application area, saturating the epidermis and limiting moisture available for fungal growth. Finally, triacetin can also be used for nutritional support whether administered orally, topically or intravenously, to restore healthy, normal skin as is seen with the topical administration of this preparation.

A concentration of 25% or greater of triacetin is a therapeutically effective concentration of triacetin utilized within the present invention. When composed of triacetin in combination with tolnaftate, the present preparation in comprised of 97 to 99.5% triacetin, and 0.5 to 1.0% tolnaftate. Alternatively, the preparation may use grisiofulvin as the antifungal agent, either by itself or in association with other antifungal agents, such as tolnaftate. In that preferred embodiment, the present preparation is comprised of 40 to 50% triacetin, 30 to 50% acetone, 0.5 to 1.0% tolnaftate and 0.5 to 4.0% grisiofulvin. When grisiofulvin is used independently, it is utilized at a concentration of 0.5 to 4.0%, in association of 40 to 50% triacetin, 30 to 50% acetone, and about 10% ethyl alcohol.

Preferably, the preparation further comprises a fatty acid source, such as cod liver oil. Cod liver oil has long been used as a nutritional supplement to limit infectious disease and for its source of vitamins A and D. One milliliter of cod liver oil contains approximately 850 IU of vitamin A and 85 IU of vitamin D within a rich source of omega-3 fatty acids. No antifungal properties have previously been associated with cod liver oil. Vitamin A in high concentrations may possess some antifungal properties by aiding the body heal itself. Cod liver oil has skin healing and softening properties similar to commercially available vitamins A and D ointments. Vitamin A and fatty acids add synergy in treating fungal infections.

Vitamin A is a fat soluble vitamin that is used in a wide dosage range from 5 to 50,000 I.U. to assist in healing. In post surgical patients, 50,000 international units applied topically to the surgical incision area improved healing time, from my experience and others. Vitamin A is also important for nutritional support. Vitamin D is also a fat soluble vitamin utilized in nutritional support. Vitamin D is not recommended in doses of over 6000 International Units per day. Topical applications with vitamin A and D have been used for years to enhance healing and repair damaged skin.

Numerous fish oils, free fatty acids, triglicerides, or omega-3 fatty acid oils can be used, ideally if they contain natural trace minerals. The oil concentration should be kept low. For example, with cod liver oil, concentrations from 0.5% to 1.0% cod liver oil have been effective. The oil concentration should be kept low due to the increased exposure of the skin to water. Many fungal infections occur with increased perspiration at the site, or in the vicinity of the infection. Use of high concentrations of oil on the skin produces a barrier around the skin, and inhibits the drying necessary for optimal healing and the desirable antifungal environment. Though 0.5% to 1.0% concentrations are effective when using cod liver oil, other more pure sources of fish oil having less barrier characteristics may be used at a concentration up to 3%, within the preparation.

Fungicidal drugs that work in vitro do not always exhibit effective results in vivo. For example, drugs like terbinafine, an antifungal agent with constant presence in nails infected by dermatophytes, needs three months or more of continuous daily intake to show some efficacy. As such, the fungicidal characteristics of terbinafine appear irrelevant in clinical practice. One reason for this is that the status of the fungal cells is quite different in vivo. The fungal cells that are dormant are not responsive to the toxic effects of most antifungal agents in use today. The dormant fungal cells are much less sensitive than hyphae to the action of any current antifungal agent. Fish oil interacts with the triacetin, the selected antifungal agent, and the affected area as follows. Fish oils or omega-3 fatty acid oils and natural trace minerals, change the biological status of the fungal cells from a dormant stage to a growing cell. The growing cells produce hyphae which are susceptible to the toxic effects of effective antifungal agents, including free fatty acids from the fish oil.

The fish oil or omega-3 fatty acid oil also provide nutritional support and healing compounds to the infected skin, aiding in skin regrowth and healing. Additional penetration of the triacetin and selected antifungal agent, into dry, cracked, hardened and/or dead tissue is seen with the addition of omega-3 fatty acids or fish oils.

When combined to create the preparation of the present invention, the fish oil acts as an enhancer for the triacetin and the antifungal agent. The triacetin lowers the pH of the surrounding environment of the fungal infection, as well as reducing the moisture content of this area. Reducing the pH and moisture levels aids in combating the fungal infection. Tolnaftate then works to inhibit the growth and/or kill the fungi infecting the skin and/or nails of the patient.

Other compounds may optionally also be included in the preparation. For example, saturated solutions of n-acetylcysteine improve absorption of tolnaftate into the skin. Other amino acids may be incorporated to aid in absorption, as well as their healing properties. N-acetylcysteine improves penetration of this preparation into the skin and nail at very low concentrations. Topical concentrations from 0.2 to 1.0% are effective. N-acetycysteine has the unique ability to increase glutathione levels in functioning, living skin cells, making them more resistant to damage from toxic substances, and apoptosis or cell death caused by toxic substances. For example cells with high glutathione levels are resistant to chemotherapy. N-acetylcysteine also has positive immune system benefits, including but not limited to improving T. cell function which is deficient in patients prone to fungal infections.

Furthermore, herbal supplements, such as garlic or garlic oil, for example, are incorporated into certain embodiments, for their skin healing and fungistatic and/or fungicidal properties. The garlic oil concentration is limited due to its odor. The mechanism of action of garlic oil is different from antifungal agents on the market today. The proposed mechanism is a direct interaction with the fungus by organic acids and volatile oils, contained within garlic oil, including organosulfur compounds, phytic acid, saponins, amino acids including arginine. Concentrations of 0.08 o 0.1% have been used to increase overall effectiveness of the preparation but use of higher concentrations is limited due to the garlic odor. This can vary with the garlic oil source and growing conditions.

Alcohol, preferably ethyl alcohol, may be used to increase the solubility of other agents of the preparation, including tolnaftate and fish oil within the triacetin solution. Alcohol also aids in combating the growth of the infecting fungi.

In a second preferred embodiment, the preparation is comprised of 40 to 50% triacetin, 30 to 50% acetone, 0.5 to 1.0% cod liver oil, 0.5 to 3.0% tolnaftate and/or 0.5 to 4.0% grisiofulvin, and 0.2 to 1.0% n-acetylcysteine. In another preferred embodiment the preparation is comprised of 40 to 50% triacetin, 30 to 50% acetone, 0.5 to 4.0% grisiofulvin and/or 0.5 to 3.0% tolnaftate, 0.5 to 1.0% cod liver oil, 0.2 to 1.0% n-acetylcysteine, and 0.08 to 0.1% garlic oil. In a further preferred embodiment, the preparation is comprised of 40 to 50% triacetin, 30 to 50% acetone, 0.5 to 1.0% cod liver oil, 0.5 to 4.0% grisiofulvin and/or 0.5 to 3.0% tolnaftate, 0.2 to 1.0% n-acetylcysteine, 0.08 to 0.1% garlic oil, and 5-15% ethyl alcohol. It should be understood that the components of these embodiments may be selectively included or removed as desired, so long as the preparation includes triacetin, a fish oil, or like fatty acid source and an antifungal agent such as tolnaftate or grisiofulvin.

In compounding the preparation of the present invention, it is important that the concentration of fish oil or like fatty acid source is not too great. For example, cod liver oil concentrations greater that 1.0% have decreased the effectiveness of this preparation. An increased concentration of fish oil retains the moisture content of the affected area, which aids in fungal growth.

Examples of other antifungal agents that can be combined with fish oils and triacetin to arrive at the preparation include polyenes such as nystatin; imidazoles such as clortimazole, econazole, ketoconazole, miconazole, solconazole, and oxiconizole; and, allylamines-benzylamines, including naftifine, terbinafine, and butenafine, for example. Other antifungal agents, whether or not presently approved by the U.S. Food & Drug Administration, may be selected and used, once approved and shown effective.

The preparation is applied topically when the infection presents itself, until the infection is removed. The preparation is applied twice daily, until the infection is resolved. For more complicated infections, a longer treatment period is needed. This preparation has shown effective results in diabetic patients, whom had previously been on oral antifungal treatments such as LAMISIL produced by Novartis AG. Clinical examples of the effectiveness of the present invention are set forth below.

Fungal infections complicated by concurrent bacterial infections or non-susceptible fungi, or in patients with systemic diseases such as diabetes, malignancy, and immune suppression such as HIV, or use of certain drugs such as steroids, chemotherapeutic agents, immune suppressants and/or antibiotics, need oral antifungal therapy today. The preparation of the present invention is directed at, and effectively treating fungal infections in patients in these compromised situations, without oral therapies.

Application of this preparation aids in healing the skin itself and treated areas that presented as originally hard, dry, cracked, tissue caused from fungal infections or other conditions, became soft, supple tissue.

Although the principles, preferred embodiments and preferred operation of the present invention have been described in detail herein, this is not to be construed as being limited to the particular illustrative forms disclosed. They will thus become apparent to those skilled in the art that various modifications of the preferred embodiments herein can be made without departing from the spirit or scope of the invention as defined by the appended claims.

What is claimed is:

1. A topical antifungal preparation comprising:
A therapeutically effective concentration of triacetin in order to the reduce moisture content of the infected tissue; and,
An antifungal agent.

2. A topical antifungal preparation as recited in claim 1 comprising:
A concentration of about 25% or greater triacetin; and,
An antifungal agent.

3. A topical antifungal preparation as recited in claim 1, wherein said antifungal agent is tolnaftate.

4. A topical antifungal preparation as recited in claim 3, wherein said preparation comprises about 40 to 50% concentration triacetin, and about 0.5 to 3.0 percent concentration tolnaftate.

5. A topical antifungal preparation as recited in claim 1, further comprising an additional antifungal agent.

6. A topical antifungal preparation as recited in claim 5, wherein said additional antifungal agent is grisiofulvin.

7. A topical antifungal preparation as recited in claim 5, further comprising acetone.

8. A topical antifungal preparation as recited in claim 7, wherein said antifungal agents are tolnaftate and grisiofulvin.

9. A topical antifungal preparation as recited in claim 8, wherein said preparation comprises about 40 to 50% concentration triacetin, 0.5 to 3.0% concentration tolnaftate, about 0.5 to 4.0% grisiofulvin, and about 30 to 50% acetone.

10. A topical antifungal preparation as recited in claim 1, wherein said antifungal agent is selected from the group consisting of nystatin, clortrimazole, econazole, ketoconazole, miconazole, solconazole, oxiconizole, naftifine, terbinifine, butenafine and grisiofulvin.

11. A topical antifungal preparation as recited in claim 5, wherein said additional antifungal agent is selected from the group consisting of nystatin, clortrimazole, econazole, ketoconazole, miconazole, solconazole, oxiconizole, naftifine, terbinifine, butenafine and grisiofulvin.

12. A topical antifungal preparation comprising:
A therapeutically effective concentration of triacetin in order to the reduce moisture content of the infected tissue;
An antifungal agent; and,
Synthetic vitamin A.

13. A topical antifungal preparation as recited in claim 12, wherein said antifungal agent is tolnaftate.

14. A topical antifungal preparation as recited in claim 12, wherein said antifungal agent is selected from the group consisting of nystatin, clortrimazole, econazole, ketoconazole, miconazole, solconazole, oxiconizole, naftifine, terbinifine, butenafine and grisiofulvin.

15. A topical antifungal preparation as recited in claim 12, further comprising an additional antifungal agent.

16. A topical antifungal preparation as recited in claim 15, wherein said additional antifungal agent is selected from the group consisting of nystatin, clortrimazole, econazole, ketoconazole, miconazole, solconazole, oxiconizole, naftifine, terbinifine, butenafine and grisiofulvin.

17. A topical antifungal preparation as recited in claim 15, further comprising acetone.

18. A topical antifungal preparation as recited in claim 17, wherein said preparation comprises about 40 to 50% concentration triacetin, about 0.5 to 3.0% concentration tolnaftate, about 0.5 to 4.0% grisiofulvin, about 0.5 to 1.0% concentration synthetic vitamin A, and about 30 to 40% acetone.

19. A topical antifungal preparation as recited in claim 12, further comprising alcohol.

20. A topical antifungal preparation as recited in claim 19, wherein said preparation comprises about 40 to 50% concentration triacetin, about 0.5 to 3.0% tolnaftate, about 0.5 to 4.0 grisiofulvin, about 0.5 to 1.0% synthetic vitamin A, about 30 to 50% acetone, and about 5.0 to 10% alcohol.
